(19) United States
(12) Patent Application Publication
(10) Pub. No.: US 2024/0285404 A1
(43) Pub. Date: Aug. 29, 2024

(54) COVER FOR PROSTHETIC VALVE EXPANSION MECHANISM

(71) Applicant: EDWARDS LIFESCIENCES CORPORATION, Irvine, CA (US)

(72) Inventors: Elazar Levi Schwarcz, Netanya (IL); Alon Ben-Yosef, Ramot Menashe (IL)

(21) Appl. No.: 18/649,749

(22) Filed: Apr. 29, 2024

Related U.S. Application Data
(63) Continuation of application No. PCT/US2022/049218, filed on Nov. 8, 2022.
(60) Provisional application No. 63/278,443, filed on Nov. 11, 2021.

(51) Int. Cl. A61F 2/24

(52) U.S. Cl. CPC A61F 2/2436; A61F 2/2418; A61F 2/243

(57) ABSTRACT

An implantable prosthetic device including a radially expandable and compressible frame having an inflow end portion and an outflow end portion. The frame including an actuator mechanism having a first frame member with a first inner bore and a second frame member with a second inner bore, the first and second frame members being spaced apart axially from one another, an actuator extending through the first and second inner bores, and a compressible cover member disposed over a portion of the actuator extending between the first and second frame members. Rotation of the actuator in a first direction causes the first and second frame members to move axially toward one another to expand the frame.

### CROSS-REFERENCE TO RELATED APPLICATIONS

The present application is a continuation of International Application No. PCT/US2022/049218, filed Nov. 8, 2022, which claims the benefit of U.S. Provisional Application No. 63/278,443, filed Nov. 11, 2021, both of which are incorporated herein by reference in their entirety.

### FIELD

The present disclosure relates to implantable, mechanically expandable prosthetic devices, such as prosthetic heart valves, and to methods and delivery assemblies for, and including, such prosthetic devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Prosthetic heart valves that rely on a mechanical actuator for expansion can be referred to as “mechanically expandable” prosthetic heart valves. Mechanically expandable prosthetic heart valves can provide one or more advantages over self-expandable and balloon-expandable prosthetic heart valves. For example, mechanically expandable prosthetic heart valves can be expanded to various diameters. Mechanically expandable prosthetic heart valves can also be compressed after an initial expansion (for example, for repositioning and/or retrieval). However, some known devices and methods can cause rotation or movement of the prosthetic valve during expansion.

Despite the recent advancements in percutaneous valve technology, there remains a need for improved transcatheter heart valves and delivery devices for such valves.

### SUMMARY

In a representative example, an implantable prosthetic device can comprise a radially expandable and compressible frame having an inflow end portion and an outflow end portion. The frame can comprise an actuator mechanism comprising a first frame member having a first inner bore and a second frame member having a second inner bore, the first and second frame members being spaced apart axially from one another, an actuator extending through the first and second inner bores, and a compressible cover member disposed over a portion of the actuator extending between the first and second frame members. Rotation of the actuator in a first direction can cause the first and second frame members to move axially toward one another to expand the frame.

In another representative example, an implantable prosthetic device can comprise a radially expandable and compressible frame having an inflow end portion and an outflow end portion. The frame can comprise an actuator mechanism comprising a first frame member having a first inner bore and a second frame member having a second inner bore, the first and second frame members being spaced apart axially from one another, an actuator extending through the first and second inner bores, and a compressible bellows member disposed over a portion of the actuator extending between the first and second frame members. Rotation of the actuator in a first direction can cause the first and second frame members to move axially toward one another to expand the frame.

In another representative example, an implantable prosthetic device can comprise a radially expandable and compressible frame having an inflow end portion and an outflow end portion. The frame can comprise an actuator mechanism comprising a first frame member having a first inner bore and a second frame member having a second inner bore, the first and second frame members being spaced apart axially from one another, an actuator extending through the first and second inner bores, and a compressible spring disposed over a portion of the actuator extending between the first and second frame members. Rotation of the actuator in a first direction can cause the first and second frame members to move axially toward one another to expand the frame.

In another representative example, an implantable prosthetic device can comprise a radially expandable and compressible frame having an inflow end portion and an outflow end portion. The frame can comprise an actuator mechanism comprising a first frame member having a first inner bore and a second frame member having a second inner bore, the first and second frame members being spaced apart axially from one another, an actuator extending through the first and second inner bores, and a compressible latticed member disposed over a portion of the actuator extending between the first and second frame members. Rotation of the actuator in a first direction can cause the first and second frame members to move axially toward one another to expand the frame.

In a representative example, a method can comprise inserting a distal end of a delivery apparatus into the vasculature of a patient, the delivery apparatus releasably coupled to a prosthetic valve via a plurality of actuator assemblies, the prosthetic valve including a frame comprising a plurality of actuation mechanisms each comprising a first frame member, a second frame member axially spaced from the first frame member, an actuator extending through the first and second frame members, and a compressible cover member disposed over a portion of the actuator extending between the first and second frame members. The method can further comprise advancing the prosthetic valve to a selected implantation site, and actuating the plurality of actuator assemblies to cause corresponding rotation of the actuators resulting in axial movement of the first and second frame members toward one another to radially expand the prosthetic valve and compress the compressible cover member, the compressible cover member coupled to the first and second frame members such that the compressible cover member does not rotate relative to the first and second frame members.

The foregoing and other objects, features, and advantages of the disclosure will become more apparent from the following detailed description, which proceeds with reference to the accompanying figures.

### BRIEF DESCRIPTION OF THE DRAWINGS

FIG. 1 is a perspective view of a prosthetic heart valve, according to one example.

FIG. 2 is a perspective view of a portion of the frame of the prosthetic heart valve of FIG. 1.

FIG. 3 is a partial side view of a delivery apparatus for a prosthetic heart valve, according to one example.

FIG. 4 is a side view of a portion of a delivery assembly comprising the prosthetic valve of FIG. 1 and a distal end portion of the delivery apparatus of FIG. 3, depicting the prosthetic valve in a radially expanded configuration.

FIG. 5 is a side view of a portion of a frame of a prosthetic heart valve including a cover member on the actuation mechanism, according to one example, the frame shown in the radially compressed configuration.

FIG. 6 is a side view of the prosthetic heart valve of FIG. 5 with the cover member shown in cross-section.

FIG. 7 is a side view of the prosthetic heart valve of FIG. 5, the frame shown in the radially expanded configuration.

FIG. 8 is a perspective view of a prosthetic heart valve, according to one example, the prosthetic heart valve shown in the radially compressed configuration.

FIG. 9 is a perspective view of the prosthetic heart valve of FIG. 8 including cover members on the actuation mechanisms.

FIG. 10 is a side view of a portion of a frame of a prosthetic heart valve including a cover member on the actuation mechanism, according to one example, the frame shown in the radially compressed configuration.

FIG. 11 is a side view of the prosthetic heart valve of FIG. 10, the frame shown in the radially expanded configuration.

FIG. 12 is a side view of a portion of a frame of a prosthetic heart valve including a cover member on the actuation mechanism, according to one example, the frame shown in the radially compressed configuration.

FIG. 13 is a side view of the prosthetic heart valve of FIG. 12, the frame shown in the radially expanded configuration.

FIG. 14 is a side view of a portion of a frame of a prosthetic heart valve including a cover member on the actuation mechanism, according to one example, the frame shown in the radially compressed configuration.

FIG. 15 is a side view of the prosthetic heart valve of FIG. 14, the frame shown in the radially expanded configuration.

### DETAILED DESCRIPTION

### General Considerations

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

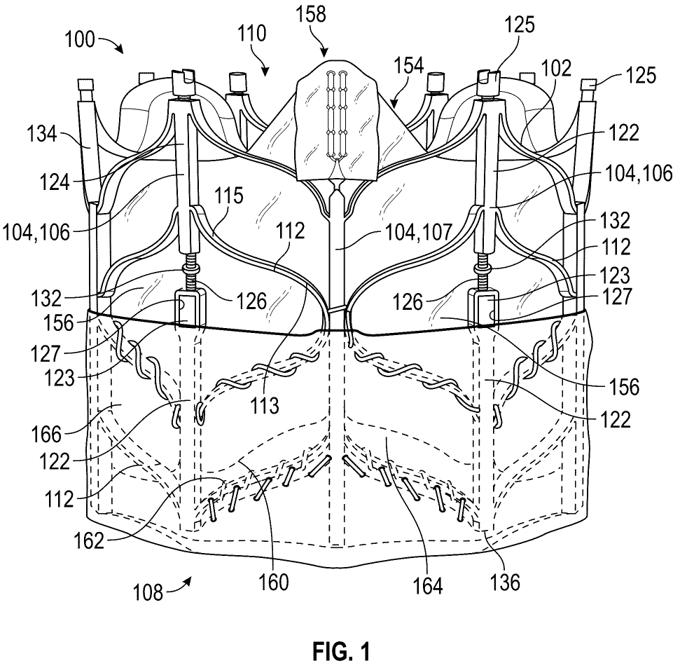

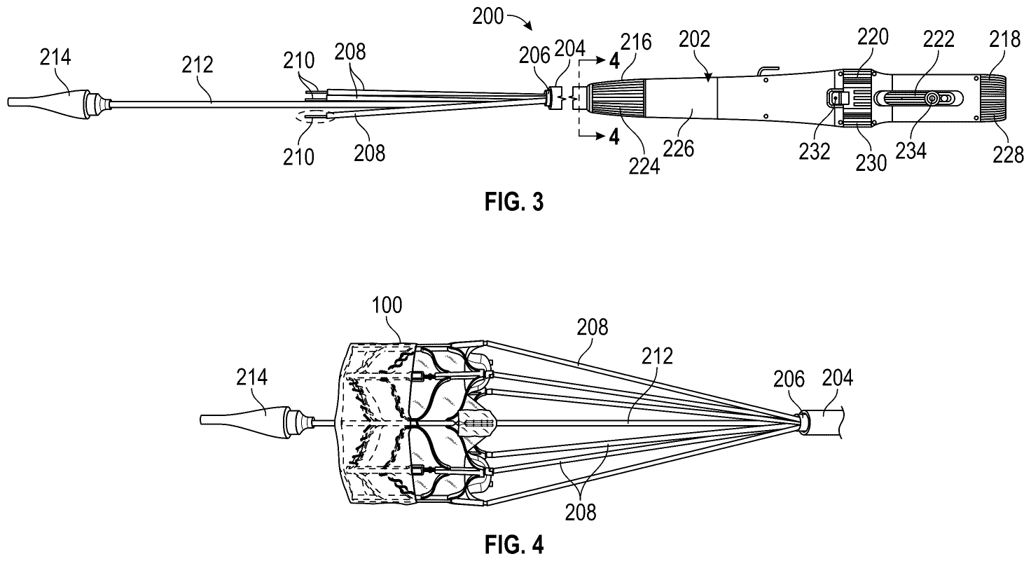

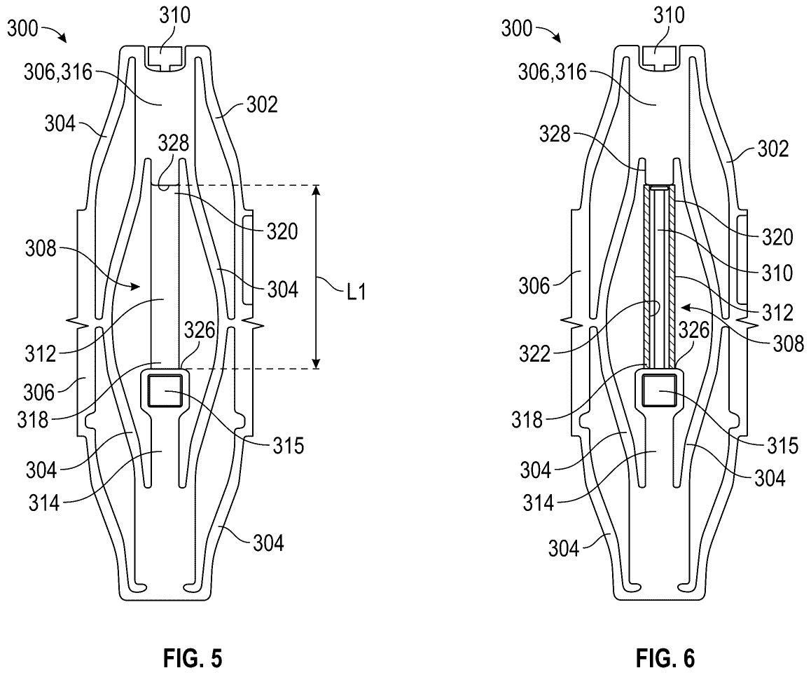

All features described herein are independent of one another and, except where structurally impossible, can be used in combination with any other feature described herein. For example, a delivery apparatus 200 as shown in FIG. 3 can be used in combination with prosthetic valves 100 or 300 described herein. In another example, cover members as shown in FIGS. 5-15 can be used in combination with the prosthetic valve 100 shown in FIG. 1.

As used in this application and in the claims, the singular forms “a,”“an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient's body), while distal motion of the device is motion of the device away from the user and toward the implantation site (for example, into the patient's body). The terms “longitudinal” and “axial” refer to an axis extending in the proximal and distal directions, unless otherwise expressly defined.

### Examples of the Disclosed Technology

Described herein are examples of prosthetic implants, such as prosthetic heart valves, that can be implanted within any of the native valves of the heart (for example, the aortic, mitral, tricuspid, and pulmonary valves). The present disclosure also provides frames for use with such prosthetic implants. The frames can further comprise actuator mechanisms (for example, expansion mechanisms) and/or locking mechanisms to enable greater control over the radial compression or expansion of the valve body. The frames can comprise struts having different shapes and/or sizes to minimize the overall crimp profile of the implant and provide sufficient structural strength and rigidity to areas where needed.

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the valves disclosed herein may be used with a variety of implant delivery apparatuses, and examples thereof will be discussed in more detail later.

FIG. 1 illustrates an exemplary example of a prosthetic valve 100 having a frame 102 including an inflow end portion 108 and an outflow end portion 110. The frame 102 can comprise a plurality of axially-extending posts 104, one or more of which can be configured as actuators 106, coupled together via a plurality of link members or struts 112. The prosthetic valve 100 can further include a valvular structure 154, which is coupled to and supported inside the frame 102. The valvular structure 154 is configured to regulate the flow of blood through the prosthetic valve 100 from the inflow end 108 to the outflow end 110.

The valvular structure 154 can include, for example, a leaflet assembly comprising one or more leaflets 156 made of flexible material. The leaflets 156 can be made from in whole or part, biological material, bio-compatible synthetic materials, or other such materials. Suitable biological material can include, for example, bovine pericardium (or pericardium from other sources). The leaflets 156 can be secured to one another at their adjacent sides to form commissures 158, each of which can be secured to a respective post 104 (for example, to a support post 107) or to the frame 102.

In the depicted example, the valvular structure 154 includes three leaflets 156, which can be arranged to collapse in a tricuspid arrangement. Each leaflet 156 can have an inflow edge portion 160. As shown in FIG. 1, the inflow edge portions 160 of the leaflets 156 can define an undulating, curved scallop edge that follows or tracks portions of the struts 112 of frame 102 in a circumferential direction when the frame 102 is in the radially expanded configuration. The inflow edges 160 of the leaflets can be referred to as a “scallop line.”

As shown in FIG. 1, the inflow edge portions 160 of the leaflets 156 can be sutured to an inner skirt 164 generally along the scallop line. The inner skirt 164 can in turn be sutured to adjacent struts 112 of the frame 102, for example, via one or more sutures 162. In other examples, the leaflets 156 can be sutured directly to the frame 102 along the scallop line.

The prosthetic valve 100 can further include one or more skirts or sealing members. For example, as mentioned previously, the prosthetic valve 100 can comprise an inner skirt 164, mounted on the radially inner surface of the frame 102. The inner skirt 164 can function as a sealing member to prevent or decrease perivalvular leakage, to anchor the leaflets to the frame, and/or to protect the leaflets against damage caused by contact with the frame during crimping and during working cycles of the prosthetic valve. The prosthetic valve 100 can further include an outer skirt 166 mounted on the outer surface of the frame 102. The outer skirt 166 can function as a sealing member for the prosthetic valve by sealing against the tissue of the native valve annulus and helping to reduce paravalvular leakage past the prosthetic valve. The inner and outer skirts 164, 166 can be formed from any of various suitable biocompatible materials, including any of various synthetic materials, including fabrics (for example, polyethylene terephthalate (PET) fabric) or natural tissue (for example, pericardial tissue). Further details regarding the use of skirts or sealing members in prosthetic valve can be found, for example, in U.S. Pat. No. 11,399,932, which is incorporated by reference herein in its entirety.

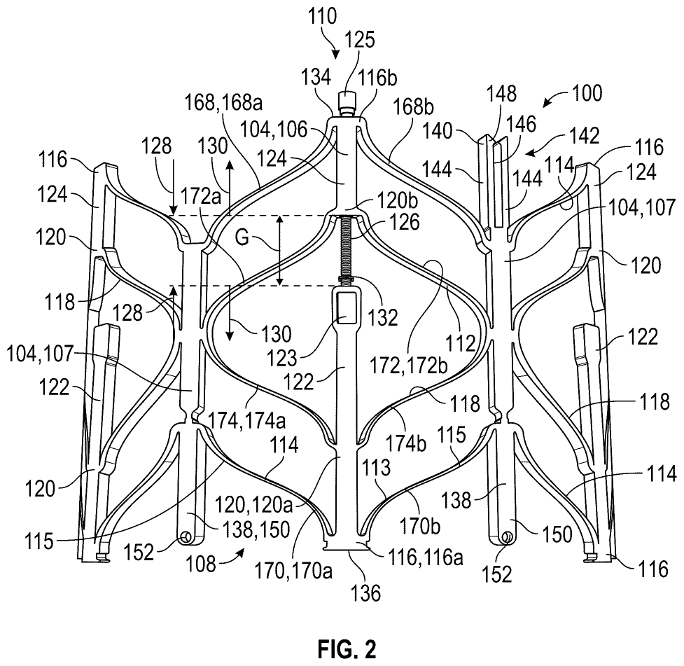

The prosthetic valve 100 can be radially expandable and compressible between a radially expanded configuration and a radially compressed configuration. FIG. 2 shows the bare frame 102 of prosthetic valve 100 (without the leaflets or other components) for purposes of illustrating the configuration of the frame 102. While only one side of the frame 102 is depicted in FIG. 2, it should be appreciated that the frame 102 forms an annular structure having an opposite side that is substantially identical to the portion shown.

As shown in FIG. 2, the struts 112 of frame 102 can comprise a curved shape. The struts 112 can define a plurality of first and second cells extending circumferentially around the frame 102. Each first cell 114 can have an axially-extending elliptical shape including first and second apices 116 (for example, inflow apex 116a and outflow apex 116b) disposed at the major vertices of the ellipse. Each first cell 114 can further comprise a respective second cell 118 disposed within the outer perimeter of the first cell 114. The second cell 118 can have a circumferentially-extending elliptical shape including first and second apices 120 (for example, inflow apex 120a and outflow apex 120b) disposed at the minor vertices of the ellipse.

In some examples, such as the example shown in FIG. 2, each strut 112 can have a recurve or S-shape including a first, upwardly curved portion 113 and a second, downwardly curved portion 115 separated by an inflection point. Each strut 112 can terminate at either end in an asymptotic manner against a post 104 such that it is nearly parallel with a longitudinal axis extending through the inflow and outflow ends of the frame 102. The struts 112 can be arranged such that the upwardly curved portion 113 is disposed nearer the inflow end 108 of the frame 102 and the downwardly curved portion 115 is disposed nearer the outflow end 110.

As mentioned, the frame 102 can comprise a plurality of axially-extending struts or posts 104, including a plurality of first struts/frame members/posts 122 (which are lower posts in the illustrated example and can extend to the inflow end of the frame) and a plurality of second struts/frame members/posts 124 (which are upper posts in the illustrated example and can extend to the outflow end of the frame). Each first post 122 can be axially aligned with a corresponding second post 124 for a pair of first and second posts. One or more pairs of posts 122, 124 can be configured as actuator mechanisms 106. The frame 102 can further comprise additional posts 104 configured as support posts 107. The support posts 107 can be disposed between each pair of adjacent circumferentially disposed first cells 114, and the actuator mechanisms 106 can be disposed such that they extend through and are coupled to the apices 116, 120 of the first and second cells. The posts 104 can be coupled together via the struts 112.

Each first cell 114 is formed by two upper struts 168a, 168b and two lower struts 170a, 170b. Each upper and lower strut 168, 170 is coupled on one end to an actuator mechanism 106 and on the other end to a support post 107. The upper struts 168a, 168b can be part of an upper row of struts that defines the outflow end of the frame and the lower struts 170a, 170b can be part of a lower row of struts that defines the inflow end of the frame. Each second cell 118 is formed by two upper struts 172a, 172b and two lower struts 174a, 174b. The lower ends of the upper struts 172a, 172b and the upper ends of the lower struts 174a, 174b can be connected to the support posts 107. The upper ends of the upper struts 172a, 172b and the lower ends of the lower struts 174a, 174b can be connected to a respective actuator mechanism 106. In the illustrated example, the upper ends of the upper struts 172a, 172b can be connected to a second post 124 and the lower ends of the lower struts 174a, 174b can be connected to a first post 122.

As mentioned, the first and second cells 114, 118 can each comprise an inflow apex 116a, 120a and an outflow apex 116b, 120b. Each pair of posts 122, 124 can extend through and be coupled to the inflow and outflow apices 116, 120 of a respective first and second cell pair. In the illustrated example, the frame 102 comprises six first cells 114 extending circumferentially in a row, with a second cell 118 within each first cell 114, and six pairs of posts 122, 124 coupled to a respective pair of cells 114, 118. However, in other examples, the frame 102 can comprise a greater or fewer number of first cells 114 within a row, and a correspondingly greater or fewer number of second cells 118 and/or pairs of posts 122, 124.

In some examples, each pair of posts 122, 124 can be configured as an actuator mechanism 106. For example, in the illustrated example, each of the six pairs of posts 122, 124 is configured as an actuator mechanism 106. In other examples, not all pairs of posts 122, 124 need be actuator mechanisms. Where a pair of posts 122, 124 is configured as an actuator mechanism, an actuator 126 (including a head portion 125) extends through each post of 122, 124 of the pair to effect radial compression and expansion of the frame. In the description that follows, the first and second posts 122, 124, respectively, that are used as actuator mechanisms (that is, those that include actuators 126), can be referred to as first and second actuator members 122, 124, or first and second frame members 122, 124. The upper end of each first frame member 122 and the lower end of a corresponding second frame member 124 can be separated by a gap G, allowing the frame members 122, 124 to move toward and away from each other during radial expansion and radial compression, respectively, of the frame. If a pair of posts 122, 124 is not used as an actuator mechanism, an actuator 126 need not extend through the posts 122, 124 of that pair.

In the illustrated example, the actuator 126 is configured as a threaded rod having an externally threaded surface. However, in other examples, the actuator 126 can be any of various members and/or mechanisms configured to move the first and second frame members 122, 124 axially relative to one another. For example, in other examples, the actuator 126 can be a linear rack comprising a plurality of teeth and configured to engage a corresponding pawl on the first and/or second frame member 122, 124, or vice versa.

Each frame member 122, 124 can comprise an inner bore extending along a length of the frame member 122, 124 and through which an actuator 126 can extend. An outflow end portion of the first frame member 122 can comprise or house a nut 123. The nut 123 can be visible through a window 127 (FIG. 1) in the outflow end portion. The nut 123 can comprise an inner threaded bore configured to engage the threads of the actuator 126 such that rotation of the actuator 126 causes the first frame member 122, which is coupled to the nut 123 to move relative to the second frame member 124, which is held steady. As shown in FIGS. 1-2, the nut 123 can be sized to fill the window 127 such that the nut 123 is restrained from movement relative to the second frame member 122.

In other examples, in lieu of using a nut 123, a portion of the inner bore of the first frame member 122 can be threaded. For example, an outflow end portion of the first frame member 122 can comprise inner threads configured to engage the actuator 126 such that rotation of the actuator causes the first frame member 122 to move relative to the second frame member 124. In still other examples, the inner bore of the first frame member 122 may be threaded along its entire length.

Rotation of the actuator 126 in a first direction (for example, clockwise) can cause corresponding axial movement of the first and second frame members 122, 124 toward one another (as shown by arrows 128), expanding the frame 102, and rotation of the actuator 126 in a second direction (for example, counterclockwise) causes corresponding axial movement of the first and second frame members 122, 124 away from one another (as shown by arrows 130), compressing the frame. As the frame 102 moves from a compressed configuration to an expanded configuration, the gap G (FIG. 2) between the first and second frame members 122, 124 of the actuator mechanism 106 can narrow.

The actuator 126 can comprise a stopper 132 (for example, a nut) disposed thereon. The stopper 132 can be disposed on the actuator 126 such that it sits within the gap G. During crimping/compression of the prosthetic valve 100, the actuator 126 can be rotated in the second direction (for example, counterclockwise) causing the stopper 132 to move toward the outflow end portion 134 of the frame 102 until it abuts the inflow edge of the second frame member 124. The stopper can thereby apply force to the second frame member 124 to cause compression of the frame 102.

Movement of the inflow end 136 and outflow end 134 of the frame 102 relative to one another to causes radial expansion or compression of the frame 102. For example, moving the inflow and outflow ends 136, 134 toward one another causes the frame 102 to foreshorten axially and expand radially. Conversely, moving the inflow and outflow ends 136, 134 away from one another causes the frame 102 to elongate axially and compress radially.

FIGS. 3-4 illustrate a delivery apparatus 200, according to one example, adapted to deliver a prosthetic valve 100, such as prosthetic valve 100 described previously. The delivery apparatus 200 can also be referred to as a “valve catheter” or a “delivery catheter.” The delivery apparatus 200 comprises a handle 202, a first shaft 204, a second shaft 206, three support sleeves 208, three actuation shafts/drivers 210, a third shaft configured as a nosecone shaft 212, and a nosecone 214. The handle 202 is configured for manipulating the various shafts and/or sleeves relative to each other. A prosthetic valve (for example, prosthetic valve 100) can be releasably coupled to the distal end portion of the delivery apparatus 200 (see, for example, FIG. 4), and the delivery apparatus 200 can be used for positioning the prosthetic valve 100, and/or for expanding, compressing, and locking the prosthetic valve 100 in a desired radially expanded configuration.

In the illustrated example, the delivery apparatus 200 comprises three pairs of support sleeves 208 and actuation shafts 210. Each support shaft 208/actuation shaft 210 pair can be referred to as an “actuator assembly.” In other examples, the delivery apparatus 200 can comprise less than three (for example, 1-2, including 1 or 2) or more than three (for example, 4-15, including 6-12, 6-9, 4, 5, 6, 7, 8, 9, 10, 11, 12, 13, 14, or 15) pairs of support sleeves 208 and actuation shafts 210, depending on the number of actuator mechanisms a prosthetic valve includes. For example, although only three pairs of support sleeves 208 and actuation shafts 210 are depicted in FIGS. 3-4 for purposes of illustration, the delivery apparatus 200 can comprise six pairs of support sleeves and actuation shafts when using the delivery apparatus 200 to implant the prosthetic valve 100 (FIGS. 1-2) since the prosthetic valve 100 comprises six actuator mechanisms.

The handle 202 of the delivery apparatus 200 can comprise one or more mechanisms configured to move the shafts and sleeves relative to each other. For example, as depicted in FIG. 3, the handle 202 comprises a deployment mechanism 216, an actuation mechanism 218, a release mechanism 220, and/or a nosecone positioning mechanism 222.

The deployment mechanism 216 of the handle 202 is coupled to the first shaft 204 and the second shaft 206 and is configured to move the first shaft 204 and the second shaft 206 axially relative to each other. The first mechanism 216 of the handle 202 can be used to deploy a prosthetic valve from a delivery capsule/delivery sheath (for example, a distal end portion) of the first shaft 204.

In the illustrated example, the deployment mechanism 216 includes a first knob 224 configured for actuating the deployment mechanism 216. In other examples, the deployment mechanism 216 can comprise various other types of actuators configured for actuating the deployment mechanism 216, such as buttons, switches, etc. The deployment mechanism 216 can also include one or more other components (such as electric motors, rotatable shafts, drive screws, gear assemblies, etc.) configured to facilitate and/or restrict relative axial movement between the first shaft 204 and the second shaft 206. For example, the deployment mechanism 216 can be configured such that rotating the first knob 224 (and/or an electric motor) relative to a housing 226 of the handle 202 results in relative axial movement between the first shaft 204 and the second shaft 206.

The actuation mechanism 218 of the handle 202 is coupled to the actuation shafts 210 and is configured to move the actuation shafts 210 axially relative to the support sleeves 208. When a prosthetic valve is coupled to the delivery apparatus 200 via the actuation shafts 210, the actuation mechanism 218 can be used to radially expand and/or compress the prosthetic valve, as further explained below. Accordingly, the actuation mechanism 218 can also be referred to as “an expansion mechanism.”

In the illustrated example, the actuation mechanism 218 comprises a second knob 228 configured for actuating the actuation mechanism 218. In other examples, the actuation mechanism 218 can comprise various other types of actuators. The actuation mechanism 218 can also include one or more additional components configured to facilitate and/or restrict relative axial movement of the actuation shafts 210 relative to the support sleeves 208. For example, the actuation mechanism 218 can comprise electric motors, drive screws, gear assemblies, and/or other components. In some examples, the actuation mechanism 218 can be configured such that rotating the second knob 228 (and/or an electric motor) relative to the housing 226 of the handle 202 results in relative axial movement between the actuation shafts 210 and the support sleeves 208.

The release mechanism 220 of the handle 202 is also coupled to the actuation shafts 210 and is configured to rotate the actuation shafts 210 relative to the support sleeves 208. In this manner, the release mechanism 220 can be used to simultaneously couple/release each of the actuation shafts 210 to/from the prosthetic valve 100. Thus, the release mechanism 220 can also be referred to as “a coupling mechanism.”

In the illustrated example, the release mechanism 220 comprises a third knob 230 configured for actuating the release mechanism 220. In other examples, the release mechanism 220 can comprise various other types of actuators. The release mechanism 220 can also comprise one or more other components (for example, a gear assembly and/or an electric motor) configured to facilitate and/or restrict relative rotational movement between the actuation shafts 210 and the support sleeves 208. For example, the release mechanism 220 can be configured such that rotating the third knob 230 relative to the housing 226 results in rotation of the actuation shafts 210 relative to the support sleeves 208. The release mechanism 220 can also comprise a lock mechanism, such as a switch 232 configured to selectively restrict rotation of the third knob 230 relative to the housing 226. In this manner, the switch 232 can prevent or reduce the likelihood that the delivery apparatus 200 is inadvertently released from a prosthetic valve.

The nosecone positioning mechanism 222 of the handle 202 is coupled to the nosecone shaft 212 and is configured to move the nosecone shaft 212 and the nosecone 214 axially relative to the first shaft 204 and the second shaft 206.

In the illustrated example, the nosecone positioning mechanism 222 comprises a slider 234 configured for actuating the nosecone positioning mechanism 222. The nosecone positioning mechanism 222 can comprise various other components configured to facilitate and/or restrict relative axial movement of the nosecone shaft 212, the first shaft 204, and the second shaft 206. For example, in some examples, the nosecone positioning mechanism 222 can comprise one or more biasing members (for example, springs) configured to bias the nosecone shaft 212 to a pre-determined axial position relative to the first shaft 204 and the second shaft 206. In such instances, the slider 234 can be biased to a particular axial position relative to the housing 226 (for example, to a proximal position). The nosecone shaft 212 can be moved axially relative to the first and second shafts by sliding the slider 234 relative to the housing 226 with sufficient force to overcome the opposing force of the biasing members. Upon release, the slider 234 can return to the biased position. In other examples, the nosecone positioning mechanism can comprise a rotatable knob, an electric motor, and/or a drive screw configured to convert relative rotational movement between the knob (and/or motor) and the housing into relative axial movement between the nosecone shaft and the first and second shafts.

The support sleeves 208 can extend distally from respective actuation lumens 240 of the second shaft 206 and can be configured to contact apices of the prosthetic valve. The support sleeves 208 can be relatively more rigid than the actuation shafts 210. As such, the support sleeves 208 can be used to apply distally-directed forces the prosthetic valve, which can oppose proximally-directed forces applied to prosthetic valve by the actuation shafts 210, thereby enabling expansion of the prosthetic valve caused by relative axial movement between the actuators and the frame of the prosthetic valve.

In the illustrated example, the support sleeves 208 are relative short tubes that are coupled to the distal end portion of the second shaft 206 but do not extend all the way through the second shaft 206 to the handle 202. The support sleeves 208 can, in some instances, be secured to the inner surfaces of the second shaft 206 that define the actuation lumens 240 (for example, via adhesive). In other examples, proximal end portions of the support sleeves 208 can be coupled to the handle 202, and the support sleeves 208 can extend through respective actuation lumens 240 of the second shaft 206 and beyond the distal end of the second shaft 206. The actuation shafts 210 can extend distally from the handle 202, through respective actuation lumens 240 of the second shaft 206, and through the lumens of respective support sleeves 208. The distal end portions of the actuation shafts 210 can comprise mating features configured to releasably couple the actuation shafts to the actuator mechanisms of the prosthetic valve. Further details of the delivery apparatus, including exemplary mating features, can be found, at least, in International Publication Nos. WO2022/251577, and WO2022/072564, which are incorporated by reference herein in their entirety.

In some examples, the actuation shafts 210 can be relatively flexible members. For example, the actuation shafts can be wires, cables, cords, sutures, etc. In other examples, the actuation shafts can be relatively rigid members, such as a rod. In other examples, the actuation shafts 210 can comprise one or more relatively flexible segments (for example, at the distal end portions) and one or more relatively rigid segments (for example, at the proximal end portions). In some examples, the delivery apparatus can further comprise an optional recompression shaft, as detailed in International Publication No. WO2022/251577.

Referring now to FIG. 4, the prosthetic valve 100 can be coupled to a distal end portion of the delivery apparatus 200 to form a delivery assembly, and the delivery apparatus 200 can be used to implant the prosthetic valve 100 within a patient's body. The prosthetic valve 100 can be coupled to the delivery apparatus 200 by positioning the delivery apparatus 200 in the configuration shown in FIG. 4. With the prosthetic valve 100 in the radially expanded configuration, the prosthetic valve 100 can be positioned over a proximal portion of the nosecone 214 and the nosecone shaft 212. The delivery apparatus can 200 can then be releasably coupled to the prosthetic valve 100.

With the prosthetic valve 100 releasably coupled to the delivery apparatus 200, the prosthetic valve 100 can be radially compressed by actuating the actuator mechanisms of the prosthetic valve (for example, such as actuator mechanisms 106 of prosthetic valve 100), by tensioning a recompression member, and/or by inserting the prosthetic valve 100 and delivery apparatus 200 into a crimping device. Once the prosthetic valve 100 is compressed, the first shaft 204 of the delivery apparatus 200 can be advanced over the second shaft 206 of the delivery apparatus 200 and the prosthetic valve 100 such that the prosthetic valve 100 is disposed within the lumen of the first shaft 204 and the distal end of the first shaft 204 abuts the nosecone 214. This can be accomplished, for example, by actuating the deployment mechanism 216 of the handle 202.

The prosthetic valve 100 can be expanded in the following exemplary manner. Generally, the prosthetic valve 100 is placed in a radially compressed state and releasably coupled to one or more actuator assemblies of a delivery apparatus 200, as described previously, and the delivery apparatus and the prosthetic valve can be advanced over a guidewire through the vasculature of a patient to a selected implantation site (for example, the native aortic annulus). For example, when implanting the prosthetic valve within the native aortic valve, the delivery apparatus and the prosthetic valve can be inserted into and through a femoral artery, and through the aorta to the native aortic valve. The prosthetic valve 100 can then be deployed at the implantation site (for example, within the native aortic valve) and can be expanded and locked in the expanded configuration using the actuator mechanisms 106. Once a selected diameter of the prosthetic valve 100 is reached, the actuator assemblies can be uncoupled from the actuator mechanisms 106 and removed from the patient's body.

To deploy the prosthetic valve 100, the physician can actuate the actuator assemblies by rotating the actuation shafts/drivers 210 in a first direction (for example, using the handle 202) which can cause corresponding rotation of the actuators 126. The rotation of the actuator 126 can cause axial movement of the first and second frame members 122, 124 of the actuator mechanism 106 toward one another to decrease the distance between the frame members 122, 124, causing the frame 102 to foreshorten axially and expand radially until a selected diameter is achieved. The disclosed actuator mechanism examples advantageously allow for continuous prosthetic valve expansion (for example, without the stepped expansion that results from a ratcheting mechanism) and allow the prosthetic heart valve to be deployed at any of various diameters.

Once the prosthetic valve 100 has been implanted at a selected implantation site within a patient, the patient's native anatomy (for example, the native aortic annulus) may exert radial forces against the prosthetic valve 100 that would tend to compress the frame 102. However, the engagement of the actuator 126 with the threaded nut 123 prevents such forces from compressing the frame 102, thereby ensuring that the frame remains locked in the desired radially expanded state.

If repositioning or recapture and removal of the prosthetic valve 100 is desired, the prosthetic valve can be compressed (from an expanded or partially expanded configuration) by rotating the actuator shafts/drivers 210 and therefore the actuators 126 in a second, opposing direction. The rotation of the actuators 126 can cause axial movement of the first and second frame members 122, 124 of the actuator mechanism 106 away from one another to increase the distance between the frame members 122, 124, causing the frame 102 to elongate axially and compress radially. Once the prosthetic valve 100 has been recompressed it can be repositioned at the implantation site, once repositioned, the prosthetic valve 100 can be expanded as described previously. The prosthetic valve can be re-compressed, repositioned, and re-expanded multiple times, as needed. In some cases, the prosthetic valve 100 can be fully compressed and “recaptured,” that is, retracted back into a sheath and/or removed from the patient's body.

Once final positioning and expansion of the prosthetic valve is achieved, the actuator assemblies can be released from the prosthetic valve 100 by retracting the support shafts 208 to uncover the connection between the actuation shafts/drivers 210 and the actuators 126. When each support shaft 208 is retracted, the actuation shaft/driver 210 can be uncoupled from a respective actuator 126 thereby uncoupling the prosthetic valve 100 from the delivery apparatus. At this stage, the delivery apparatus 200 can be retracted relative to the prosthetic valve 100 and removed from the patient's body.

FIGS. 5-15 illustrate another example of a prosthetic valve 300. Prosthetic valve 300 can be similar to prosthetic valve 100 (for example, including a frame 302 having a plurality of struts 304 that couple one or more axially-extending posts 306, one or more of which are configured as actuator mechanisms 308 including an actuator 310) except that prosthetic valve 300 further comprises a compressible sleeve/cover member 312. While FIGS. 5-15 depict only a portion of the frame 302 of prosthetic valve 300, it should be appreciated that the frame 302 forms an annular structure similar to frame 102 described previously. It should further be appreciated that prosthetic valve 300 can comprise a valvular structure (such as valvular structure 154 described earlier) coupled to and supported inside the frame, and inner and/or outer skirts, as previously described, although these components are omitted for purposes of illustration.

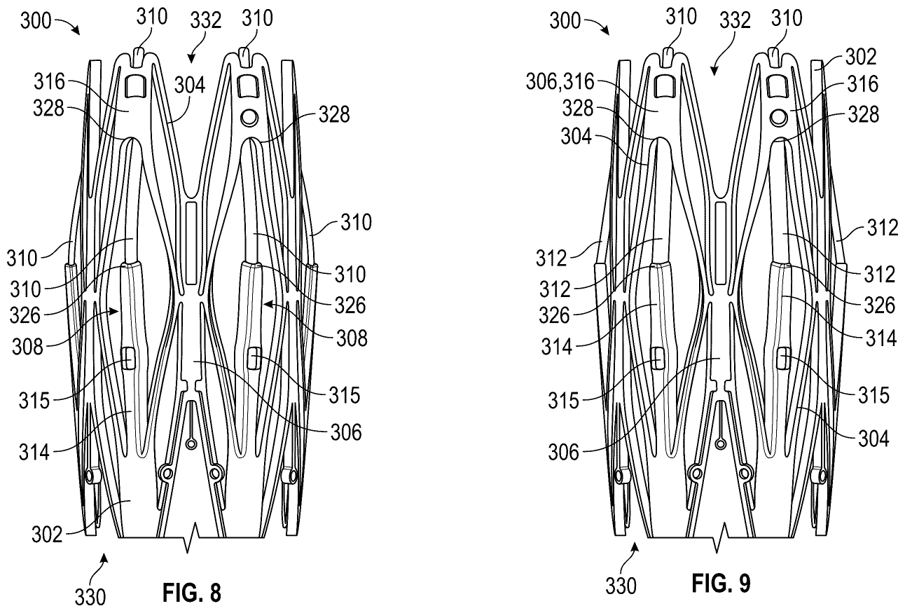

The compressible cover member 312 can be configured to prevent direct contact of the leaflets of the valvular structure (for example, valvular structure 154 described previously) with the actuator/actuation bolt/threaded rod 310. In some examples, as discussed with respect to FIGS. 8-9, the cover member 312 can further provide additional structural support to the actuator 310 to prevent buckling and/or deformation of the actuator 310 during crimping and/or expansion of the prosthetic valve 300.

Referring to FIGS. 5-7, the compressible cover member 312 can be disposed between the first frame member 314 (which can comprise a nut 315 similar to nut 123 described previously) and the second frame member 316 of the actuator mechanism 308. The cover member 312 can have a first end portion 318 and a second end portion 320 and can comprise an inner lumen or bore 322 (FIG. 6) through which at least a portion of the actuator 310 can extend. The bore 322 can be sized such that the actuator 310 does not contact an inner surface of the bore 322 when the actuator 310 is rotated (for example, during expansion and/or compression of the prosthetic valve 300).

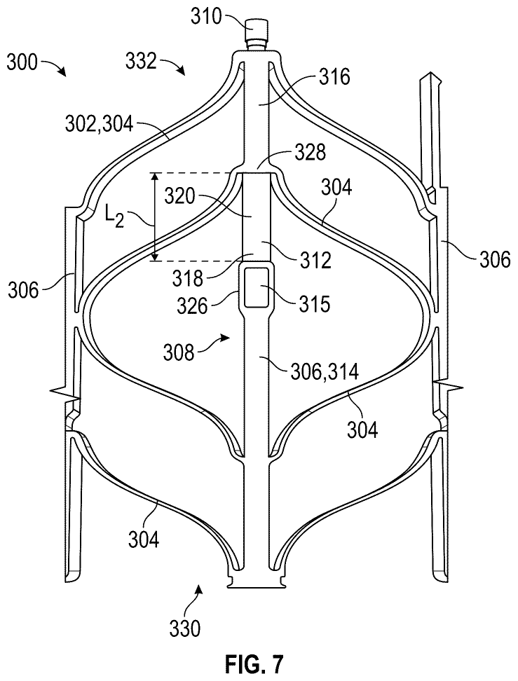

The cover member 312 can be axially compressible and expandable between a first, expanded length L₁ when the frame 302 is in a radially compressed configuration (FIG. 5), and a second, compressed length L₂ when the frame 302 is in a radially expanded configuration (FIG. 7). In some examples, such as the illustrated example, the expanded length L₁ of the cover member 312 can extend over the entire portion of the actuator 310 between the first and second frame members 314, 316 (also referred to as the “exposed portion” of the actuator 310). Such a configuration advantageously prevents or mitigates the leaflets of the valvular structure from directly contacting the actuator 310, thereby preventing the leaflets from being wrapped around the actuator and potentially damaged as the actuator is rotated. In other examples, the cover member 312 can have an expanded length L₁ that extends only partially over the exposed portion of the actuator 310. In the illustrated example, the cover member 312 has a generally cylindrical shape, however, in other examples, the cover member can have, for example, a conical shape, a barrel shape, and/or an hourglass shape.

In some examples, the cover member 312 can be configured to expand and/or compress multiple times (for example, can be resilient) such that it can return to its original expanded shape if the prosthetic valve is re-compressed. In other examples, the cover member 312 can be configured to be compressed a single time.

In some examples, such as the illustrated example, the first end portion 318 of the cover member 312 can be coupled to an outflow end portion 326 of the first frame member 314 and the second end portion 320 can be coupled to an inflow end portion 328 of the second frame member 316, such that the cover member 312 is restrained against rotation relative to the frame 302. In other examples, the cover member 312 can “float” between the first frame member 314 and the second frame member 316. In some examples, the cover 312 can additionally include one or more anti-rotation features (for example, protrusions/projections received within corresponding recesses in the first and/or second frame members 314, 316) configured to restrain the cover 312 against rotation about its longitudinal axis.

In some examples, as shown in FIGS. 5-7, the cover member 312 can be configured as a sleeve. The sleeve can comprise a stretchable and/or elastic material, such as a polymeric material, for example, polyethylene and/or Nylon.

Referring to FIGS. 8-9, the cover member 312 can be configured to bend and/or deform if the frame 302 assumes a non-cylindrical configuration (for example, a barrel shape or a V-shape) such as during crimping of the frame 302. In some examples, when the frame 302 is crimped/compressed, the frame 302 can assume a non-cylindrical configuration, such as a barrel configuration wherein the inflow and outflow end portions 330, 332 of the frame 302 have a diameter smaller than that of a central portion 334 of the frame 302. As shown in FIG. 8, crimping of the frame 302 into a barrel configuration can sometimes result in deformation/buckling/bending of the exposed portion of the actuator 310. If the actuators 310 are bent or otherwise deformed, they may be unable to rotate in order to expand and/or compress the frame 302.

As shown in FIG. 9, the cover member(s) 312 can provide additional structural support to the actuator(s) 310 to prevent and/or mitigate unintentional deformation/buckling/bending. In such examples, the cover members 312 can extend fully from the outflow end portion 326 of the first frame member 314 to the inflow end portion 328 of the second frame member 316. In such examples, the cover member 312 can have a flexural modulus higher than that of the actuator 310. In other words, the cover member 312 can be stiffer/less bendable than the actuator 310. The cover member 312 can have an outer diameter greater than an outer diameter of the actuator 310, such that, though the cover member 312 can be axially flexible with a flexural modulus lower than that of the actuator 310, the cover member 312 has a higher resistance to buckling relative to the actuator 310.

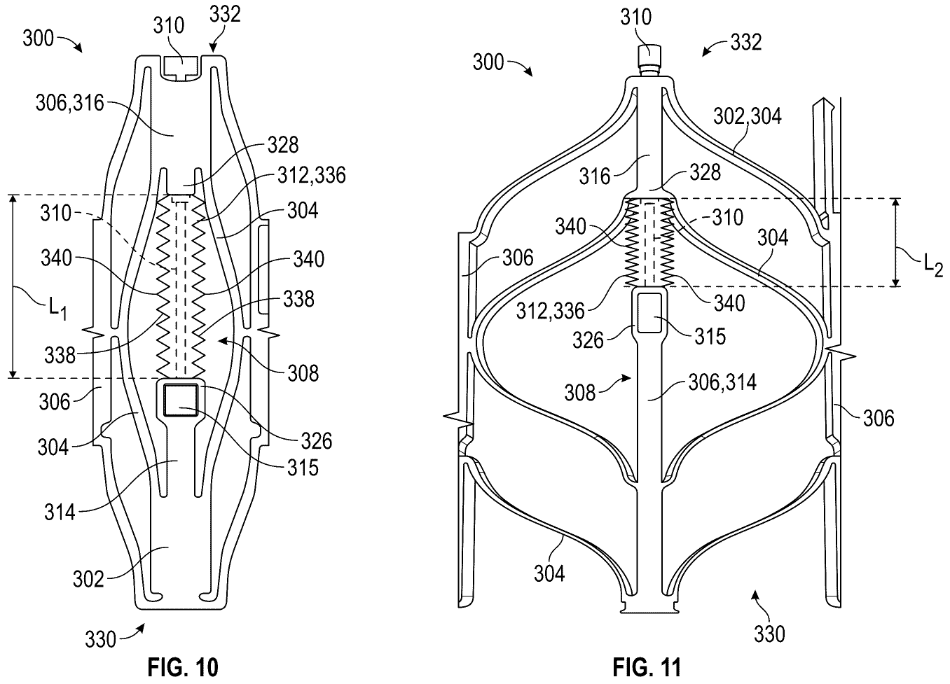

Referring to FIGS. 10-11, in some examples, the cover member 312 can be configured as a compressible bellows member 336 such as a sleeve bellows or expansion joint. The bellows member 336 can have a generally cylindrical shape including an inner bore through which at least a portion of the actuator 310 extends. The bellows member 336 can comprise a plurality of ridges or folds 338, each having an apex 340. The bellows member 336 can be axially compressible and expandable between a first, expanded length L₁ when the frame 302 is in a radially compressed configuration (FIG. 10), and a second, compressed length L₂ when the frame 302 is in a radially expanded configuration (FIG. 11). The axial distance between the apices 340 of the folds 338 can be greater when the bellows member 336 is in the expanded configuration than when the bellows member is in the compressed configuration. In other words, as the bellows member 336 is compressed the apices 340 move closer together.

In some examples, such as the illustrated example, the expanded length L₁ of the bellows member 336 can extend over the entire exposed portion of the actuator 310 and be coupled to the outflow end portion 326 of the first frame member 314 and the inflow end portion 328 of the second frame member 316 such that the bellows member 336 does not rotate relative to the frame 302. Such a configuration advantageously prevents or mitigates the leaflets of the valvular structure from directly contacting the actuator 310, thereby preventing the leaflets from being wrapped around the actuator and potentially damaged when the actuator rotates. Rather, the leaflets can contact the relatively atraumatic surface of the bellows member 336 instead. In other examples, the bellows member 336 can have an expanded length L₁ that extends only partially over the exposed portion of the actuator 310.

In some particular examples, the bellows member 336 can comprise a polymeric material for example, polyethylene and/or Nylon. In other examples, the bellows member 336 can comprise a flexible metal (such as stainless steel), and/or an elastomer (such as rubber).

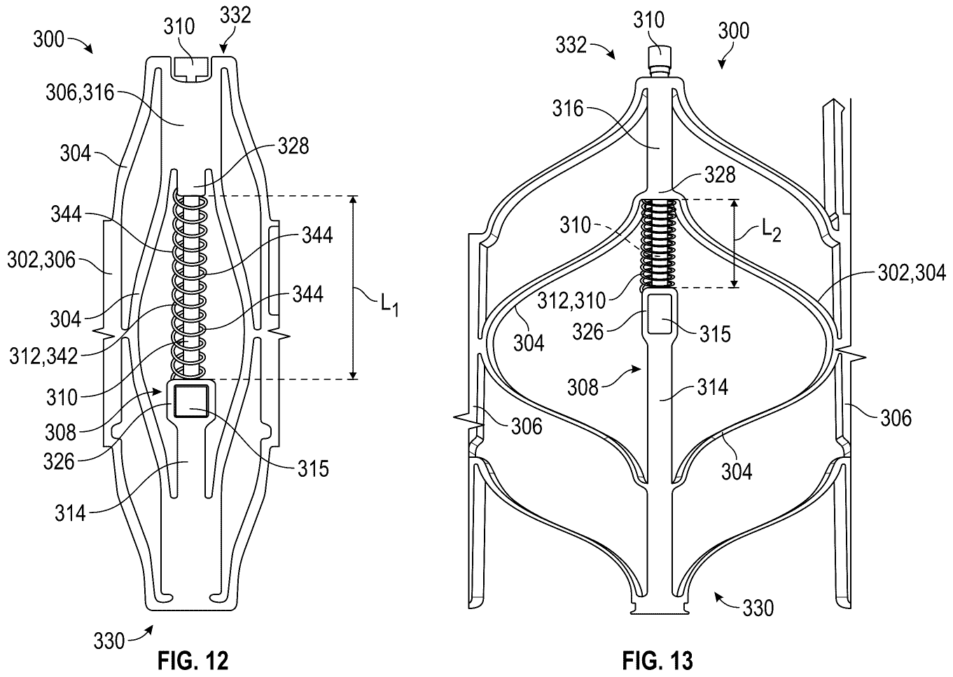

Referring to FIGS. 12-13, in some examples, the cover member 312 can be configured as a compressible spring 342 (such as a compression spring) including an inner bore through which the actuator 310 extends. In the illustrated example, the compressible spring 342 has a generally cylindrical shape, however, in other examples, the spring can have a conical shape, a barrel shape, and/or an hourglass shape.

The spring 342 can comprise a plurality of coils 344 and can be axially compressible and expandable between a first, expanded length L₁ when the frame 302 is in a radially compressed configuration (FIG. 12), and a second, compressed length L₂ when the frame 302 is in a radially expanded configuration (FIG. 13). The axial distance between the coils 344 can be greater when the spring 342 is in the expanded configuration than when the spring is in the compressed configuration. In other words, as the spring 342 is compressed the coils 344 move closer together. The spring 342 can be biased into the expanded configuration, for example, by shape-setting.

In some examples, such as the illustrated example, the expanded length L₁ of the spring 342 can extend over the entire exposed portion of the actuator 310 and be coupled to the outflow end portion 326 of the first frame member 314 and the inflow end portion 328 of the second frame member 316 such that the spring 342 does not rotate relative to the frame 302. Such a configuration advantageously prevents or mitigates the leaflets of the valvular structure from directly contacting the actuator 310, thereby preventing the leaflets from being wrapped around the actuator and potentially damaged when the actuator rotates. Rather, the leaflets can contact the relatively atraumatic non-rotating coils of the spring 342 instead.

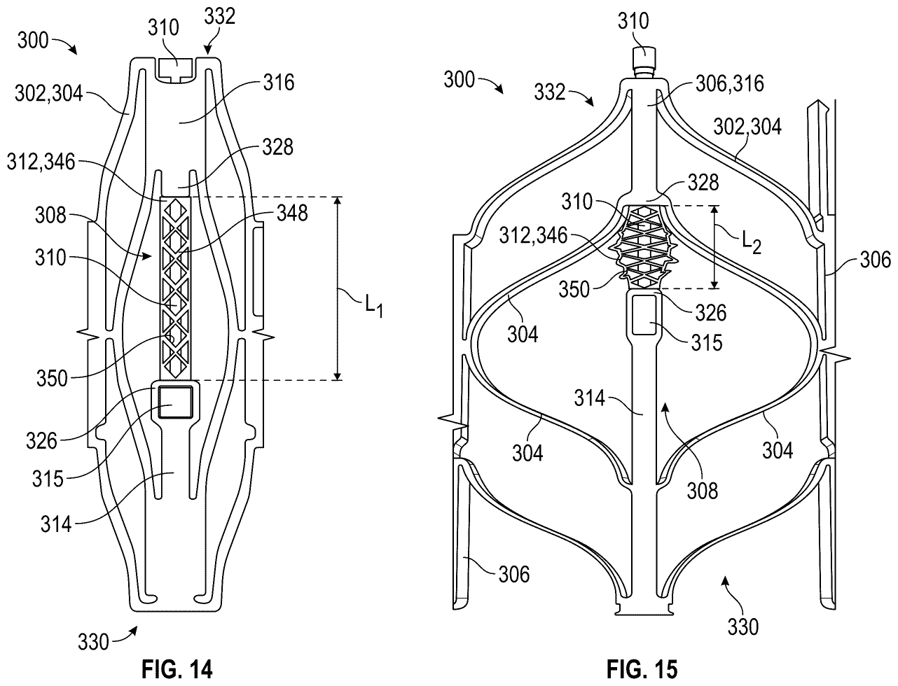

Referring to FIGS. 14-15, in still other examples, the cover member 312 can be configured as a latticed member 346 (such as a laser-cut hypotube) including an inner bore through which at least a portion of the actuator 310 extends. The latticed member 346 can comprise a plurality of struts 348 defining a plurality of cells 350. The latticed member 346 can be axially compressible and expandable between a first, expanded length L₁ when the frame 302 is in a radially compressed configuration (FIG. 14), and a second, compressed length L₂ when the frame 302 is in a radially expanded configuration (FIG. 15). The axial distance between the apices of the cells 350 can be greater when the latticed member 346 is in the expanded configuration than when in the compressed configuration. In other words, as the latticed member 346 is compressed the struts 348 can deform such that the apices of the cells 350 move closer together.

In the illustrated example, the latticed member 346 has a generally cylindrical shape in the expanded configuration, however, in other examples, the latticed member 346 can have a conical shape, a barrel shape, and/or an hourglass shape. The shape of the latticed member 346 can be selected to adjust the tube's ability to resist axial compression and/or to adjust the flexural modulus of the tube (for example, to allow or resist lateral bending of the latticed member).

In some examples, such as the illustrated example, the expanded length L₁ of the latticed member 346 can extend over the entire exposed portion of the actuator 310 and be coupled to the outflow end portion 326 of the first frame member 314 and the inflow end portion 328 of the second frame member 316 such that the latticed member 346 does not rotate relative to the frame 302. Such a configuration advantageously prevents or mitigates the leaflets of the valvular structure from directly contacting the actuator 310, thereby preventing the leaflets from being wrapped around the actuator and potentially damaged when the actuator rotates. Rather, the leaflets can contact the relatively atraumatic surface of the latticed member 346 instead.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An implantable prosthetic device, comprising:
a radially expandable and compressible frame having an inflow end portion and an outflow end portion, the frame comprising:
an actuator mechanism comprising:
a first frame member having a first inner bore and a second frame member having a second inner bore, the first and second frame members being spaced apart axially from one another,
an actuator extending through the first and second inner bores, and
a compressible cover member disposed over a portion of the actuator extending between the first and second frame members; and
wherein rotation of the actuator in a first direction causes the first and second frame members to move axially toward one another to expand the frame.

Example 2. The prosthetic device of any example herein, particularly example 1, wherein the actuator comprises an external threaded surface.

Example 3. The prosthetic device of any example herein, particularly example 2, further comprising a nut disposed at an outflow end portion of the second frame member, the nut comprising an internal threaded surface configured to engage the external threaded surface of the actuator.

Example 4. The prosthetic device of any example herein, particularly any one of examples 1-3, wherein rotation of the actuator in a second direction causes the first and second frame members to move axially away from one another to radially compress the prosthetic device.

Example 5. The prosthetic device of any example herein, particularly any one of examples 1-4, further comprising a stopper disposed on the actuator axially between the first and second frame members, the stopper configured to selectively abut an inflow end portion of the first frame member.

Example 6. The prosthetic device of any example herein, particularly any one of examples 1-5, wherein the cover member is movable between an axially expanded configuration having a first length and an axially compressed configuration having a second, shorter length.

Example 7. The prosthetic device of any example herein, particularly example 6, wherein the cover member is biased into the expanded position.

Example 8. The prosthetic device of any example herein, particularly any one of examples 1-7, wherein a first end portion of the compressible cover is coupled to the first frame member and wherein a second end portion of the compressible cover is coupled to the second frame member.

Example 9. The prosthetic device of any example herein, particularly any one of examples 1-8, wherein the compressible cover member comprises a polymeric material.

Example 10. The prosthetic device of any example herein, particularly example 9, wherein the polymeric material comprises at least one of polyethylene and Nylon.

Example 11. The prosthetic device of any example herein, particularly any one of examples 1-10, wherein the cover member further comprises one or more anti-rotation features configured to engage corresponding anti-rotation features on the frame to prevent rotation of the cover member about its longitudinal axis.

Example 12. The prosthetic device of any example herein, particularly any one of examples 1-11, wherein the cover member has a cylindrical shape including an inner bore.

Example 13. The prosthetic device of any example herein, particularly any one of examples 1-11, wherein the cover member has at least one of a conical shape, a barrel shape, and an hourglass shape.

Example 14. The prosthetic device of any example herein, particularly any one of examples 1-13, wherein the cover member has a flexural modulus higher than a flexural modulus of the actuator.

Example 15. The prosthetic device of any example herein, particularly any one of examples 1-14, wherein the cover member comprises a bellows member.

Example 16. The prosthetic device of claim 15, wherein the bellows member comprises a plurality of folds each having an apex.

Example 17. The prosthetic device of any example herein, particularly any one of examples 15-16, the bellows member comprising at least one of a polymeric material, a flexible metal, and an elastomer.

Example 18. The prosthetic device of any example herein, particularly any one of examples 1-14, wherein the cover member comprises a spring.

Example 19. The prosthetic device of any example herein, particularly any one of examples 1-14, wherein the cover member comprises a latticed member.

Example 20. The prosthetic device of any example herein, particularly example 19, wherein the latticed member comprises a plurality of struts defining a plurality of cells.

Example 21. An implantable prosthetic device, comprising:
a radially expandable and compressible frame having an inflow end portion and an outflow end portion, the frame comprising:
   an actuator mechanism comprising:
   a first frame member having a first inner bore and a second frame member having a second inner bore, the first and second frame members being spaced apart axially from one another,
   an actuator extending through the first and second inner bores, and
   a compressible bellows member disposed over a portion of the actuator extending between the first and second frame members; and
wherein rotation of the actuator in a first direction causes the first and second frame members to move axially toward one another to expand the frame.

Example 22. The prosthetic device of any example herein, particularly example 21, wherein the bellows member is movable between an expanded configuration having a first length and a compressed configuration having a second, shorter length.

Example 23. The prosthetic device of any example herein, particularly any one of examples 21-22, wherein the bellows member comprises a plurality of folds.

Example 24. The prosthetic device of any example herein, particularly any one of examples 21-23, wherein the actuator comprises an external threaded surface.

Example 25. The prosthetic device of any example herein, particularly example 24, further comprising a nut disposed at an outflow end portion of the second frame member, the nut comprising an internal threaded surface configured to engage the external threaded surface of the actuator.

Example 26. The prosthetic device of any example herein, particularly any one of examples 21-25, wherein rotation of the actuator in a second direction causes the first and second frame members to move axially away from one another to radially compress the prosthetic device.

Example 27. The prosthetic device of any example herein, particularly any one of examples 21-26, further comprising a stopper disposed on the actuator axially between the first and second frame members, the stopper configured to selectively abut an inflow end portion of the first frame member.

Example 28. The prosthetic device of any example herein, particularly any one of examples 21-27, wherein a first end portion of the bellows member is coupled to the first frame member and wherein a second end portion of the bellows member is coupled to the second frame member.

Example 29. An implantable prosthetic device, comprising:
a radially expandable and compressible frame having an inflow end portion and an outflow end portion, the frame comprising:
   an actuator mechanism comprising:
   a first frame member having a first inner bore and a second frame member having a second inner bore, the first and second frame members being spaced apart axially from one another,
   an actuator extending through the first and second inner bores, and
   a compressible spring disposed over a portion of the actuator extending between the first and second frame members; and
wherein rotation of the actuator in a first direction causes the first and second frame members to move axially toward one another to expand the frame.

Example 30. The prosthetic device of any example herein, particularly example 29, wherein the spring is movable between an expanded configuration having a first length and a compressed configuration having a second, shorter length.

Example 31. The prosthetic device of any example herein, particularly any one of examples 29-30, wherein the spring comprises a plurality of coils.

Example 32. The prosthetic device of any example herein, particularly any one of examples 29-31, wherein the actuator comprises an external threaded surface.

Example 33. The prosthetic device of any example herein, particularly example 32, further comprising a nut disposed at an outflow end portion of the second frame member, the nut comprising an internal threaded surface configured to engage the external threaded surface of the actuator.

Example 34. The prosthetic device of any example herein, particularly any one of examples 29-33, wherein rotation of the actuator in a second direction causes the first and second frame members to move axially away from one another to radially compress the prosthetic device.

Example 35. The prosthetic device of any example herein, particularly any one of examples 29-34, further comprising a stopper disposed on the actuator axially between the first and second frame members, the stopper configured to selectively abut an inflow end portion of the first frame member.

Example 36. The prosthetic device of any example herein, particularly any one of examples 29-35, wherein a first end portion of the spring is coupled to the first frame member and wherein a second end portion of the spring is coupled to the second frame member.

Example 37. The prosthetic device of any example herein, particularly any one of examples 29-36, wherein the spring has a cylindrical shape including an inner bore.

Example 38. The prosthetic device of any example herein, particularly any one of examples 29-37, wherein the spring has at least one of a conical shape, a barrel shape, and an hourglass shape.

Example 39. An implantable prosthetic device, comprising:
a radially expandable and compressible frame having an inflow end portion and an outflow end portion, the frame comprising:
   an actuator mechanism comprising:
   a first frame member having a first inner bore and a second frame member having a second inner bore, the first and second frame members being spaced apart axially from one another,
   an actuator extending through the first and second inner bores, and
   a compressible latticed member disposed over a portion of the actuator extending between the first and second frame members; and
wherein rotation of the actuator in a first direction causes the first and second frame members to move axially toward one another to expand the frame.

Example 40. The prosthetic device of any example herein, particularly example 39, wherein the latticed member is movable between an expanded configuration having a first length and a compressed configuration having a second, shorter length.

Example 41. The prosthetic device of any example herein, particularly any one of examples 39-40, wherein the latticed member is a laser-cut hypotube.

Example 42. The prosthetic device of any example herein, particularly any one of examples 39-41, wherein the latticed member comprises a plurality of struts defining a plurality of cells.

Example 43. The prosthetic device of any example herein, particularly any one of examples 39-42, wherein the actuator comprises an external threaded surface.

Example 44. The prosthetic device of any example herein, particularly example 43, further comprising a nut disposed at an outflow end portion of the second frame member, the nut comprising an internal threaded surface configured to engage the external threaded surface of the actuator.

Example 45. The prosthetic device of any example herein, particularly any one of examples 39-44, wherein rotation of the actuator in a second direction causes the first and second frame members to move axially away from one another to radially compress the prosthetic device.

Example 46. The prosthetic device of any example herein, particularly any one of examples 39-45, further comprising a stopper disposed on the actuator axially between the first and second frame members, the stopper configured to selectively abut an inflow end portion of the first frame member.

Example 47. A method, comprising:
inserting a distal end of a delivery apparatus into the vasculature of a patient, the delivery apparatus releasably coupled to a prosthetic valve via a plurality of actuator assemblies, the prosthetic valve including a frame comprising a plurality of actuation mechanisms each comprising a first frame member, a second frame member axially spaced from the first frame member, an actuator extending through the first and second frame members, and a compressible cover member disposed over a portion of the actuator extending between the first and second frame members;
advancing the prosthetic valve to a selected implantation site; and
actuating the plurality of actuator assemblies to cause corresponding rotation of the actuators resulting in axial movement of the first and second frame members toward one another to radially expand the prosthetic valve and compress the compressible cover member, the compressible cover member coupled to the first and second frame members such that the compressible cover member does not rotate relative to the first and second frame members.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. An implantable prosthetic device, comprising:
a radially expandable and compressible frame having an inflow end portion and an outflow end portion, the frame comprising:
an actuator mechanism comprising:
a first frame member having a first inner bore and a second frame member having a second inner bore, the first and second frame members being spaced apart axially from one another,
an actuator extending through the first and second inner bores, and
a compressible cover member disposed over a portion of the actuator extending between the first and second frame members; and
wherein rotation of the actuator in a first direction causes the first and second frame members to move axially toward one another to expand the frame.

2. The prosthetic device of claim 1, wherein the actuator comprises an external threaded surface.

3. The prosthetic device of claim 2, further comprising a nut disposed at an outflow end portion of the second frame member, the nut comprising an internal threaded surface configured to engage the external threaded surface of the actuator.

4. The prosthetic device of claim 1, wherein rotation of the actuator in a second direction causes the first and second frame members to move axially away from one another to radially compress the prosthetic device.

5. The prosthetic device of claim 1, further comprising a stopper disposed on the actuator axially between the first and second frame members, the stopper configured to selectively abut an inflow end portion of the first frame member.

6. The prosthetic device of claim 1, wherein the cover member is movable between an axially expanded configuration having a first length and an axially compressed configuration having a second, shorter length.

7. The prosthetic device of claim 6, wherein the cover member is biased into the expanded position.

8. The prosthetic device of claim 1, wherein a first end portion of the compressible cover is coupled to the first frame member and wherein a second end portion of the compressible cover is coupled to the second frame member.

9. The prosthetic device of claim 1, wherein the compressible cover member comprises a polymeric material.

10. The prosthetic device of claim 9, wherein the polymeric material comprises at least one of polyethylene and Nylon.

11. The prosthetic device of claim 1, wherein the cover member further comprises one or more anti-rotation features configured to engage corresponding anti-rotation features on the frame to prevent rotation of the cover member about its longitudinal axis.

12. The prosthetic device of claim 1, wherein the cover member has a cylindrical shape including an inner bore.

13. The prosthetic device of claim 1, wherein the cover member has at least one of a conical shape, a barrel shape, and an hourglass shape.

14. The prosthetic device of claim 1, wherein the cover member has a flexural modulus higher than a flexural modulus of the actuator.

15. The prosthetic device of claim 1, wherein the cover member comprises a bellows member.

16. The prosthetic device of claim 15, wherein the bellows member comprises a plurality of folds each having an apex.

17. The prosthetic device of claim 15, the bellows member comprising at least one of a polymeric material, a flexible metal, and an elastomer.

18. The prosthetic device of claim 1, wherein the cover member comprises a spring.

19. The prosthetic device of claim 1, wherein the cover member comprises a latticed member and wherein the latticed member comprises a plurality of struts defining a plurality of cells.

20. A method, comprising:
inserting a distal end of a delivery apparatus into a vasculature of a patient, the delivery apparatus releasably coupled to a prosthetic valve via a plurality of actuator assemblies, the prosthetic valve including a frame comprising a plurality of actuation mechanisms each comprising a first frame member, a second frame member axially spaced from the first frame member, an actuator extending through the first and second frame members, and a compressible cover member disposed over a portion of the actuator extending between the first and second frame members;
advancing the prosthetic valve to a selected implantation site; and
actuating the plurality of actuator assemblies to cause corresponding rotation of the actuators resulting in axial movement of the first and second frame members toward one another to radially expand the prosthetic valve and compress the compressible cover member, the compressible cover member coupled to the first and second frame members such that the compressible cover member does not rotate relative to the first and second frame members.
